(19) United States
(12) Patent Application Publication
(10) Pub. No.: US 2016/0175250 A1
(43) Pub. Date: Jun. 23, 2016

(54) Method for Making Liposomes Containing an Active Pharmaceutical Ingredient

(71) Applicant: Tolmar Inc., Fort Collins, CO (US)

(72) Inventors: John Milton Downing, Fort Collins, CO (US); Stephen David Hilpert, Fort Collins, CO (US)

(73) Assignee: Tolmar Inc., Fort Collins, CO (US)

(21) Appl. No.: 14/926,482

(22) Filed: Oct. 29, 2015

Related U.S. Application Data
(60) Provisional application No. 62/096,153, filed on Dec. 23, 2014.

(51) Int. Cl. A61K 9/127; A61K 31/704

(52) U.S. Cl. CPC A61K 9/1277; A61K 31/704

(57) ABSTRACT

A method for size reduction of liposomes that may be used for administration of a drug into the body of an individual in which the size reduction method is by homogenization alone without the use of extrusion.

This application claims priority from pending U.S. Provisional Patent Application Ser. No. 62/094,153, filed on Dec. 23, 2014.

### FIELD OF THE INVENTION

This application pertains to the field of manufacturing liposomes, and particularly to the field of manufacturing liposomes that contain within an active pharmaceutical ingredient (API).

### BACKGROUND OF THE INVENTION

Liposomes are microscopic hollow spheres (vesicles) of one or more lipid. bilayers arranged concentrically and radially around an aqueous core. The lipid bilayer is composed primarily of phospholipids, molecules that have a “head” portion that is hydrophilic and a “tail” portion that is hydrophobic. When formed in an aqueous solution, the hydrophilic and hydrophobic regions determine the alignment of the phospholipid molecules m the lipid bilayer of a liposome membrane. Within each layer, the tails line up together on one side with the hydrophilic heads on the other, making the tail side: hydrophobic and the head side hydrophilic.

In the bilayer membrane, the two layers are aligned with hydrophobic (tail) sides facing each other and the hydrophilic (head) sides facing outward. When the bilayer is wrapped around to form a hollow, spherical vesicle (liposome), this molecular arrangement provides liposomes with a hydrophilic outer surface, which facilitates hydration of blended lipid solids during liposome formation in aqueous fluids, as well as a hydrophilic interior surface that forms a cavity in which may be dissolved water-soluble compounds. Additionally, liposomes provide a hydrophobic portion within the bilayer itself.

Liposomes may be used as carriers for delivery of drugs. Lipophilic drugs are sequestered within the lipid bilayer of the liposome. Hydrophilic drugs are sequestered within the aqueous core of the liposome. Amphiphilic drugs may in most cases he sequestered within a liposome at the interface between the lipid bilayer and the aqueous core.

The pharmacokinetics of any drug enclosed in liposomes is dependent on the behavior of the liposomes in the blood. An important control factor for the behavior of liposomes in blood, particularly the liposomal pharmacokinetics and the distribution of the liposome and drug in tissues, is the particle (liposome) diameter and size distribution of the liposomes in which the drug, is contained. Because these parameters are of critical importance for liposome preparations, and because liposome preparation, techniques produce heterogeneous mixtures of liposome sizes, a distinct particle diameter regulation step is utilized in the liposome manufacturing process. This step is one of the most important steps in the production of liposome preparations, especially for liposomes intended as drug carriers.

Liposome size regulation includes both a reduction in the average size of liposomes in a population and a reduction in the overall size distribution of the liposomes. In order to accomplish these goals, size regulation of liposomes is accomplished by the use of either or both of extrusion or homogenization.

As used herein, the term “extrusion” refers to a process in which a material, such as a liposome-containing composition, is forced through a membrane containing orifices in order to reduce liposomes to target size. When performed under pressure, the liposome composition is forced through the membrane, with liposomes that are larger than the pores undergoing plastic deformation as they pass through the pore and shear upon leaving the pore. The sheared-off portion that has passed through the pore spontaneously reforms into a liposome of smaller diameter.

As used herein, the term “homogenization” refers to a size reduction process other than extrusion in which a material, such as a liposome-containing composition, is made to be similar in size throughout. Examples of homogenization methods useful for liposome size regulation include use of high-shear forces, such as by a rotor/stator mill use of induced cavitation via pressure differential, such as by a piston gap; and sonication (ultrasound).

Doxorubicin is a widely used antineoplastic drug, that has a broad spectrum of reactivity and has excellent antineoplastic activity against a number of human cancer diseases. It is commonly used to treat some leukemias, Hodgkin's lymphoma, as well as cancers of the bladder, breast, stomach, lung, ovaries, thyroid, soft tissue sarcoma, and multiple myeloma.

Toxicities encountered with the administration of doxorubicin include myelosuppression, alopecia, mucositis, and gastrointestinal toxicities including nausea, vomiting and anorexia. The most serious doxorubicin toxicity is cumulative dose dependent irreversible cardiomyopathy leading to congestive heart failure in 1-10% of patients receiving doses greater than 550 mg per square meter of body area. However, it has been established that the therapeutic index (the ratio of the amount of drug that causes the therapeutic effect to the amount that causes toxicity) of doxorubicin cancer therapy employing antineoplastic agents is significantly improved by encapsulating doxorubicin in liposomes. Compared to the free drug, liposome entrapped doxorubicin has a lower degree of cardiomyopathy, and its antitumor activity is also not affected when compared to free drug.

DOXIL® (doxorubicin HCl liposome injection) (Janssen Biotech, Inc., Horsham, Pa.), is indicated for the treatment of patients with ovarian cancer whose disease has progressed or recurred after platinum-based chemotherapy, for AIDS-related Kaposi's Sarcoma, and for multiple myeloma in combination with bortezomib. However, DOXIL® is often used to treat other cancers in addition to those for which it has received FDA (Food and Drug Administration) approval, including those listed in the above discussion of doxorubicin.

Doxorubin hydrochloride, as approved by the FDA as DOXIL®, is encapsulated in liposomes. Additionally, in order to reduce the rapid opsonization and clearance by macrophages of the administered doxorubicin-containing liposomes, the DOXIL® liposomes are coated with polyethylene glycol (PEG), which increases the hydrophilicity of the liposome surface and provides a steric barrier against opsonization. These resulting long-circulating liposomes, often referred to as “stealth” or “sterically stabilized” liposomes, are still removed from the blood circulation, but this removal occurs at a much lower rate and with less involvement of the hepatosplenic macrophages. Consequently, they circulate in the bloodstream for a prolonged period of time, enabling, their extravasation into solid tumors and sites of inflammation. This so-called enhanced permeability and retention (EPR) effect allows for increased local drug concentrations in the target regions. Clinical studies have shown that doxorubicin-loaded stealth liposomes prolong circulation in the blood.

In 2009, Ortho-Biotech Products, L.P., which company subsequently became Janssen Biotech, Inc., submitted a Citizen Petition to the FDA concerning DOXIL®. The Citizen Petition disclosed that, in manufacturing the doxorubicin-loaded liposomes, unloaded (lacking doxorubicin) liposomes with as PEG coating are formed in an aqueous ammonium sulfate solution. The liposome suspension is then subjected to size regulation. After size regulation, the liposome suspension is dialyzed to remove external ammonium sulfate solution, leaving only encapsulated (internal) ammonium sulfate solution, and thus creating a chemical gradient between the exterior and interior of the liposome. Doxorubicin is then added in-solution to the exterior medium of the liposomes, and crosses the membrane to the interior of the liposome as a result of the chemical gradient. This method of preparation, wherein a drug is introduced into liposomes after manufacture of the liposomes is often referred to as “remote loading.”

The Citizen Petition compared doxorubicin-loaded liposomes that had been size regulated by extrusion and those that had been size regulated by homogenization techniques. The liposomes prepared by the two methods of size regulation had a distinctly different particle size distribution, in spite of the fact that the mean particle diameter of the two preparations was similar.

As stated in the Citizen Petition, “While liposomes prepared using extrusion had a relatively narrow unimodal distribution, those produced by homogenization showed a bimodal distribution with the main fraction having a mean diameter of approximately 60 nm and a broader distribution than results from extrusion, and a secondary fraction having a mean diameter of approximately 540 nm.” The Citizen Petition then states that, “these studies illustrate that manufacturing liposomal product with different equipment could result in materials with very different physicochemical characteristics.”

Kale, et al, “Effect of Size Reduction Techniques on Doxorubicin Hydrochloride Loaded Liposomes,” International Journal of Biological & Pharmaceutical Research, 3(3):308-316 (2012), confirmed these findings regarding size regulation by extrusion or homogenization. Kate compared size reduction of liposomes by extrusion through a combination of polycarbonate filters of various pore sizes and by homogenization using a piston-gap homogenizer at a pressure of 1000 bar and 6 passes.

Kale disclosed that, even though the average sizes of Liposomes prepared by extrusion and by homogenization were similar, the size reduction obtained by extrusion was better than that obtained by homogenization. Size reduction by extrusion produced a unimodal distribution of Liposomes, whereas homogenization produced a bimodal distribution. Of greater significance was the finding that size reduction by extrusion produced Liposomes that had a lower polydispersity index (PDI), indicating a narrower size distribution, than did size reduction by homogenization, Kale concluded that the more uniform size of liposomes produced using extrusion method as compared with those produced by homogenization would improve the kinetics of liposomes inside the body.

Thus, the state of the art prior to the present application is that methods for size reduction of liposomes that include extrusion provide a narrower size distribution than do those IS that rely solely upon homogenization. In the case of doxorubicin, homogenization without extrusion is insufficient to provide as suitable homogeneity of liposome size because, even if the average particle size obtained by extrusion and by homogenization is the same, the size distribution obtained by extrusion is superior to that obtained by homogenization without extrusion.

### BRIEF DESCRIPTION OF THE DRAWINGS

FIGS. 1A, 1B, and 1C are a series of graphs that show size reduction parameters versus homogenization time. Figure IA shows average diameter over time. FIG. 1B shows MN over time. FIG. 1C shows %T over time.

FIGS. 2A, 28, and 2C are a series of graphs that show size reduction parameters versos homogenization tune with variations in homogenization pressure. FIG. 2A shows average diameter over time with varying homogenization pressure. FIG. 28 shows PDI over time with varying homogenization pressure. FIG. 2 shows Percent Transmittance (%T, a quantitative measurement of homogenate transparency) over time with varying homogenization pressure.

FIG. 3 is a graph that shows %T over time for liposome suspensions subjected to homogenization pressures of either 8,000 psi or 10,000 psi.

FIGS. 4A and 48 are a set of graphs that shows the size distribution of liposomes following homogenization at a pressure of 10,000 psi. FIG. 4A shows size distribution obtained after 10 minutes. FIG. 4B shows size distribution obtained after 180 minutes.

### DETAILED DESCRIPTION OF THE INVENTION

It has unexpectedly been discovered that, when performing size reduction of liposomes solely by homogenization techniques employing at pressure differential, the pressure at which the method is performed is a result-effective variable. It has been unexpectedly discovered that an optimum range of pressure exists in which performance of size reduction yields superior homogenization with a narrow size distribution. Pressures above and below this optimum range of pressure yield inferior homogenization, and importantly have a wider size distribution.

This unexpected discovery is in direct contrast to the prior art that discloses that liposomes that are drug-loaded, and particularly those that are doxorubicin-loaded, and that are size reduced by homogenization alone, without extrusion, are not suitable for administration, and particularly intravenous administration, to patients.

Thus, the method of this application utilizes homogenization alone to obtain size reduction of liposomes to be drug-loaded, such as doxorubicin-loaded, and eliminates the need for extrusion sizing. This eliminates process challenges, such as multiple passes through different pore size membranes, that are associated with extrusion sizing of liposomes.

Liposomes that are suitable for the present application may he made by any method by which liposomes for administration to as patient, and particularly for intravenous administration, are made. In a preferred embodiment, lipid components that are first dissolved in an organic solvent are then, following after removal of the solvent, added as a blended solid to an aqueous solution of ammonium sulfate at an elevated temperature. As the lipid blend hydrates in this solution, liposomes spontaneously form. Subsequent size reduction processing is performed after liposome formation.

Liposomes suitable for the present application may also have a PEG coating or other suitable coating that increases EPR. The coating can be achieved by chemically bonding PEG to one of the phospholipid components before liposome formation.

After the liposomes have been made, the liposomes are size reduced in order to obtain a narrow particle size distribution and, preferably, a unimodal size distribution. According to the method of this application, the size reduction of the liposomes is achieved by homogenization techniques and without the use of extrusion.

Examples of suitable homogenization techniques include the use of shear forces such as by rotor/stator milling, the use of a pressure differential-induced cavitation, such as by a piston gap homogenizer, and sonication. hi a preferred embodiment, size reduction of liposomes is by the use of a pressure differential, and most preferably by piston gap homogenization.

In a preferred embodiment, drug to be loaded into the liposomes, such as doxorubicin, is added to the liposomes following the size reduction of the liposomes, a process referred to as “remote loading” of liposomes. Before remote loading, the ammonium sulfate solution external to the liposomes is replaced with a sucrose solution through a process such as diafiltration resulting in a chemical gradient across the liposomal bilayer, with ammonium sulfate on the inside but not on the outside of the liposomes. The drug in solution is combined with the suspension of liposomes, after which the drug passes through the liposome membrane to chemically bond with the sulfate ion, thus loading the liposome or, conversely, encapsulating, the drug. Loading is performed at elevated temperature to increase the permeability of the lipid bilayer by increasing the mobility of lipids within the liposome membrane, thereby facilitating passage of ions and drug through the membrane. In this way, drug is loaded into the liposomes in exchange for the ammonium ion, which passes out to the exterior of the liposome.

Alternatively, but less preferred, a drug, such as doxorubicin, may be loaded into the liposomes before size reduction of liposomes. This method is less preferred because breakage and reassembly of liposomes occur during homogenization, which may result in release and loss of loaded drug from liposomes into the suspension mixture.

Size reduction by use of pressure differential-induced cavitation is preferably performed by piston gap homogenization. In this process, a suspension containing liposomes is forced by means of a pump through a small gap formed between an air pressure-regulated piston and a flat surface (seat). Because of the large diameter difference between the suspension-holding compartment and the piston gap, when the suspension is forced through the gap, the dynamic pressure rises and the static pressure lid is according to Bernoulli's Law. As a result of the static pressure falling below the vapor pressure of the liquid in the suspension, the liquid begins to boil in the homogenization gap, which results in the formation of gas bubbles. These gas bubbles implode and collapse after leaving the gap (cavitation) where the pressure is again under a normal atmospheric pressure of 1 bar. This cavitation, together with shear threes and particle collision that occur as the suspension is forced through the small gap, results in disintegration of the liposomes and spontaneous re-formation into smaller liposomes.

The piston gap homogenization process may be by discrete-pass processing (DPP), in which the entire volume of liposome-containing suspension is sent through the homogenization chamber in a series of discrete, discontinuous passes. This method has several disadvantages due to the necessity to suspend processing while the sample is collected and set-up for the subsequent pass.

More preferred than DPP is continuous-loop processing (CLP) in which the liposome suspension is continuously recirculated between a reservoir vessel, and the homogenizer. Between the reservoir and the homogenizer, a Percent Transmittance (%T) measuring device is situated to determine when the desired sizing has been obtained. The CLP process permits the maintenance of continuous pressure, temperature, and flow rate throughout the treatment process.

Theoretically, DPP is more efficient than CLP because, with DPP, all particles of the Liposome suspension go through the treatment the same number of times whereas, with CLP, the mixing effect of recirculation necessarily means that, statistically, some particles will remain in the reservoir longer than others. This means that some particles pass through the homogenizer more times than other particles. However, in practice, the theoretical lesser efficiency of CLP is of little or no consequence because equivalent degrees of size reduction can be achieved by extending processing time.

The duration of homogenization processing can be described either by number of passes (for DPP) or by total time (for CLP). The amount of equivalent passes can be calculated from total CLP processing time by dividing the total CLP time by the time required for one DPP pass, that is, the time needed for the entire suspension quantity to pass once through the homogenizing chamber. For example, if one pass takes 10 minutes, 40 minutes of CLP processing is approximately equal to 4 DPP passes.

After size-reduction processing for a time, it has been observed that size characterizing parameters, such as average diameter, polydispersity index (PDI), and D₁₀-D₅₀-D₉₀ values, as obtained by a measuring device (such as a laser light scattering particle size analyzer), become asymptotic with respect to time. This suggests that, after reaching the asymptotic region of the aforementioned parameters, no further significant size changes with respect to the liposome population can be achieved with additional processing. However, this assumption has been shown to be erroneous by empirical filtration studies and utilization of a more sensitive analytical technique, that is, measurement of homogenate transparency (%T) by a device such as a UV/Vis spectrophotometer. Increased filtration yields with additional homogenization processing is evidence of continued size reduction over time. In addition, the %T vs. time plot continues to change after corresponding plots of PDI, average diameter and D₁₀-D₅₀-D₉₀ values have reached asymptotic flat lines, indicating that further change does occur in the homogenate.

Even %T and time-indexed filtration studies have indicated, however, that narrowing the size distribution after a certain amount of processing becomes increasingly difficult, requiring increasingly more processing time to achieve measurable change. For example, it has been assumed that because DPP processing achieves asymptotic PDI values after 4-6 passes, no more significant size reduction occurs. Although this assumption is erroneous, the general principle holds true that further size reduction requires increasingly longer processing times (or number of passes).

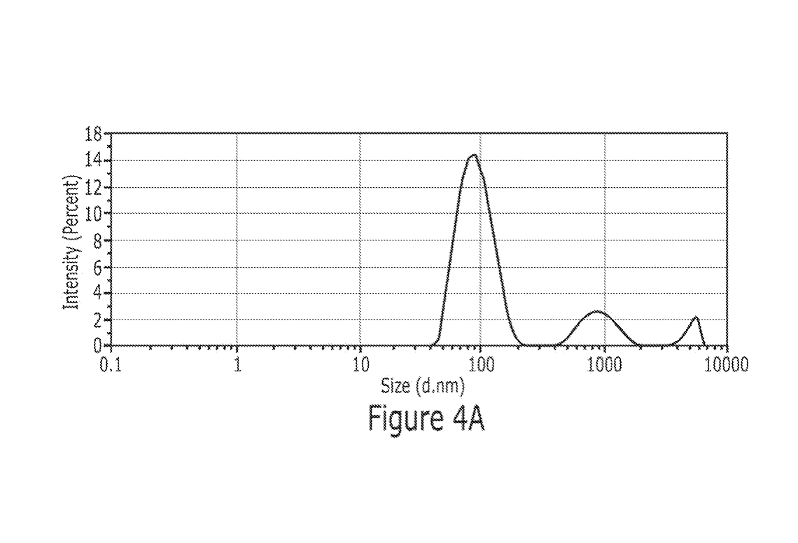

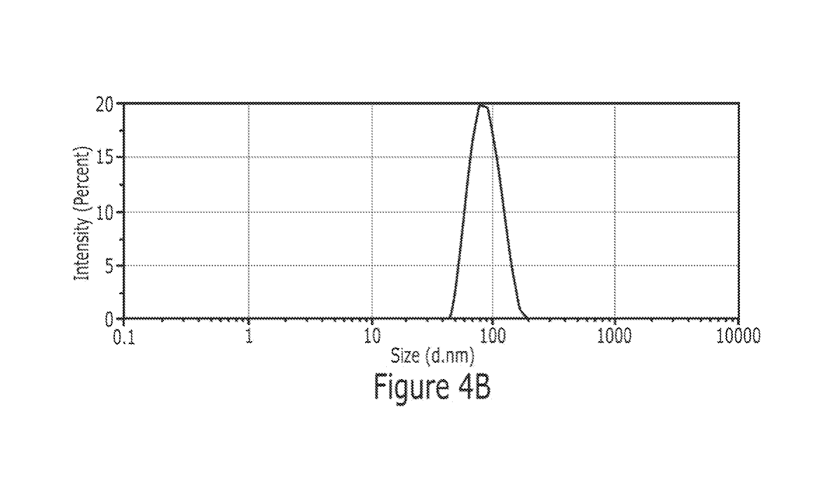

It has been discovered that an optimized size distribution of liposomes, and even a uni-modal size distribution, may be obtained when performing homogenization employing a suitable pressure differential. This is shown graphically in FIG. 4B. The piston gap homogenization pressure that is suitable for the present application is between 5000 psi (345 bar) and 12,000 psi 1827 bar). A preferred range of pressures is between 7500 psi (517 bar) and 11,000 psi (758 bar), with a most preferred pressure of 10,000 psi (690 bar). It has been unexpectedly discovered that, below 5000 psi and above 12,000 psi, the optimization of parameters pertaining to size reduction, and particularly the polydispersity index, precipitously declines. In this application, when using the term “about” with reference to homogenization pressure, this indicates plus or minus 500 psi (34.5 bar).

The measurement of homogenate transmittance over time (%T) may be used to indicate changes in liposome particle size distribution, which can be correlated to changes in the polydispersity index of a liposome sample or lot. This is because, when sizes of liposomes in a sample are more uniform, that is “less polydisperse”, the transmittance of light through the sample increases. That is, transparency of the liposome sample increases as overall particle size decreases and as size distribution narrows. Thus, not only are %T and average size reciprocally related, but %T and PDI are also reciprocally related.

When size reducing liposomes within a suspension, liposomes made from different lots of lipid components may display different absolute %T with the same amount of homogenization processing, and yet exhibit equivalent PDI. However, although the %T vs. time profiles of different lipid blend lots may vary in magnitude, all lots exhibit the same basic plot shape, which may he used to determine the end point for homogenization processing.

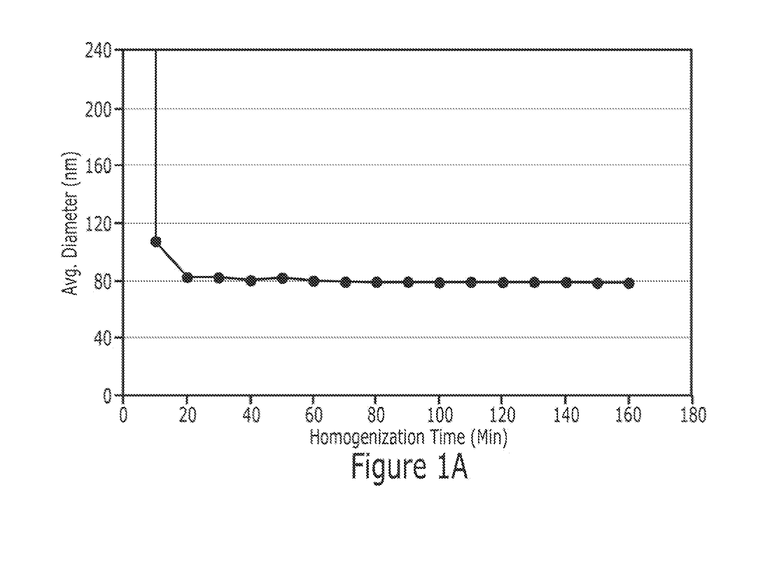

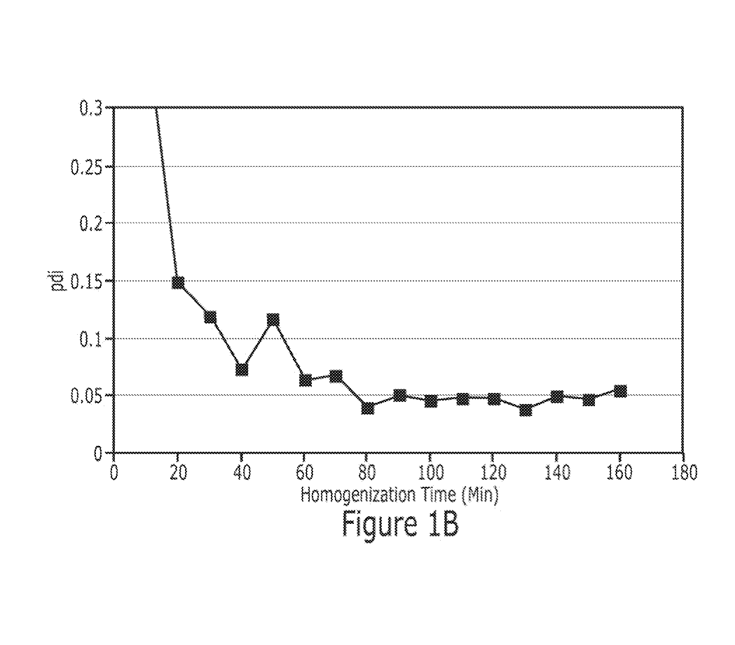

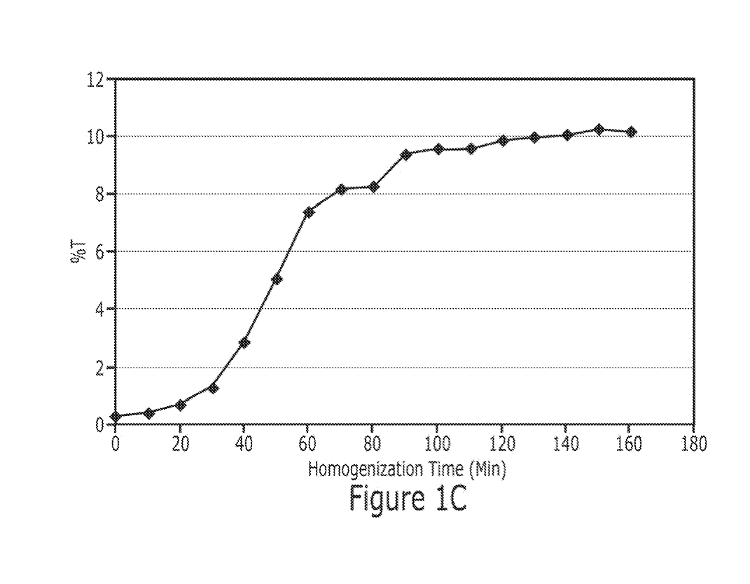

FIGS. 1A, 1B, and 1C show curves of transmittance (%T), average size, and polydispersity (PDI) during processing, either by DPP or CLP methods. As indicated in FIG. 1C, when size reducing a liposome suspension, the %T profile typically goes through four sections that correspond to four hypothesized stages in liposome size reduction. In the first stage, transparency (%T) increase is slight and gradual, indicating that large particles are still present in sufficient numbers to significantly inhibit light transmittance. During this first stage, both PDI, as shown in FIG. 1B, and average diameter, as shown in FIG. 1A, rapidly decrease, which indicates that the majority of the particle size reduction takes place during this stage.

In the second stage, there is a rapid and large increase in transparency. This is accompanied by a reciprocal rapid and large decrease in polydispersity. The average diameter becomes asymptotic in an essentially flat-line curve. Any additional significant increase in transparency by further processing is thus due, not to reduction of average diameter, but to reduction in dispersity of size of particles around the average a narrowing of particle size distribution). The rapid increase in %T during this stage indicates a rapid reduction in the number and overall diameter of the largest particles.

In the third stage, the %T curve is characterized by an inflection or shoulder region that indicates a slowing rate of increase. This coincides with a similar, although opposite, flattening of the PDI curve into a slowly decreasing, that is narrowing, of particle size distill⁻Anion. The flattening of the %T and PDI curves indicates that the liposome suspension contains a greatly decreased population of larger sized particles that exert a correspondingly decreased impact on overall transparency and PDI, and the “reduced efficiency” in locating individual large particles as they become more and more difficult to “find” in the suspension, and as the difference between average and largest sizes grows smaller.

In the fourth stage, the %T plot continues to rise, but in a more linear fashion at a much reduced slope, indicating that additional particle, size reduction is occurring at a reduced rate. In contrast, the PDI plot becomes asymptotic (essentially flat) because the degree of further particle size reduction is too small to be precisely quantified duo to limitations of the measuring device, such as a laser light scattering particle sizer. This indicates that near the end of the homogenizing process, %T can be a more sensitive measuring technique for indicating additional, though gradual and subtle, particle size change than PDI determined by particle-size measuring instruments, such as a laser light scattering particle size analyzer.

The invention is further illustrated in the following examples.

### Example 1

Manufacture of Blank Liposomes

A lipid blend containing (a) N-(carbamoyl-O-methyl-polyethylen glycol 2000)-1,2-distearoyl-sn-glycero-3-phosphtidyl-ethanolamine salt (“MPEG 2000-DPSE”), (b) hydrogenated soy phosphotidyl choline (“HSPC”), and (c) cholesterol USP was obtained from Lipoid GmbH (Newark, N.J.). In a 10 L glass reactor, 330 grams of the lipid blend was “dissolved” (hydrated) with agitation in 8.2 kg of hot (˜62° C.) 250 mM Ammonium Sulfate, NF (Hawkins, Inc., Roseville, Minn.) solution. The solution was stirred at ˜100 RPM with as rotating paddle blade for 60 minutes to form a suspension multi-membrane layer liposomes (multi-lamellar vesicles or MLVs) with diameters larger than target size.

### Example 2

Size Reduction Parameters Over Time

The MLV suspension of Example 1 was passed through a high-pressure homogenizer to change the MLVs to unilamellar liposome vesicles and to reduce liposome diameter to target size (average and overall distribution).

The MLV liposome suspension was subjected to CLP processing for size reduction in an EmulsiFlex-C55 Homogenizer (Avestin, Inc., Ottawa, Ontario, Canada). Size and distribution parameters of average size and PDI were determined by laser light scattering using a Malvern Zetasizer NS (Malvern Instruments Ltd., Malvern, Worcestershire, UK). The liposome suspension was homogenized at 62° C., with 10000 psi of pressure. Percent transmittance of the suspension was measured at 500 nm using a Thermo Scientific Genesis 20 spectrometer (Thermo Fisher Scientific Inc., Waltham, Mass.). The results are shown in FIG. 1A, 1B, and 1C.

As shown in FIG. 1A, the average diameter decreased rapidly during processing and, by 20 minutes, has reached a flat, stable level. Following this time, only very minor decreases in average diameter are obtained, regardless of duration of additional size reduction processing.

As further shown in FIG. 1B and FIG. 1C, polydispersity (PDI) and transparency (%T) are inversely correlated. The decrease in PDI initially occurs rapidly, and then slows, finally reaching a flat-line at a steady slate. In direct contrast, %T initially increases slowly, then increases rapidly, reaching a shoulder stage, and then a stage where increases in %T occur very slowly over time.

### Example 3

Size Reduction Parameters with Change in Homogenizing Pressure

Pegylated liposome suspensions prepared according to Example 1 were subjected to CLP processing, according to Example 2, except that homogenization pressure was varied during treatment.

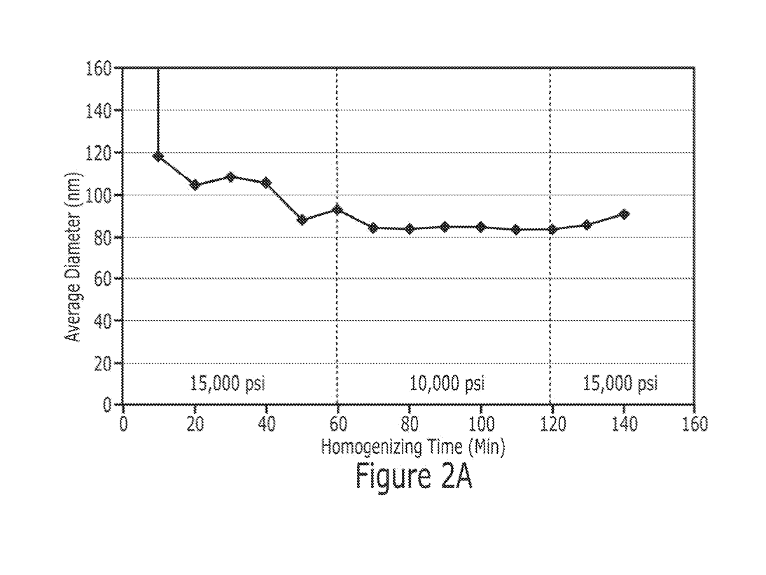

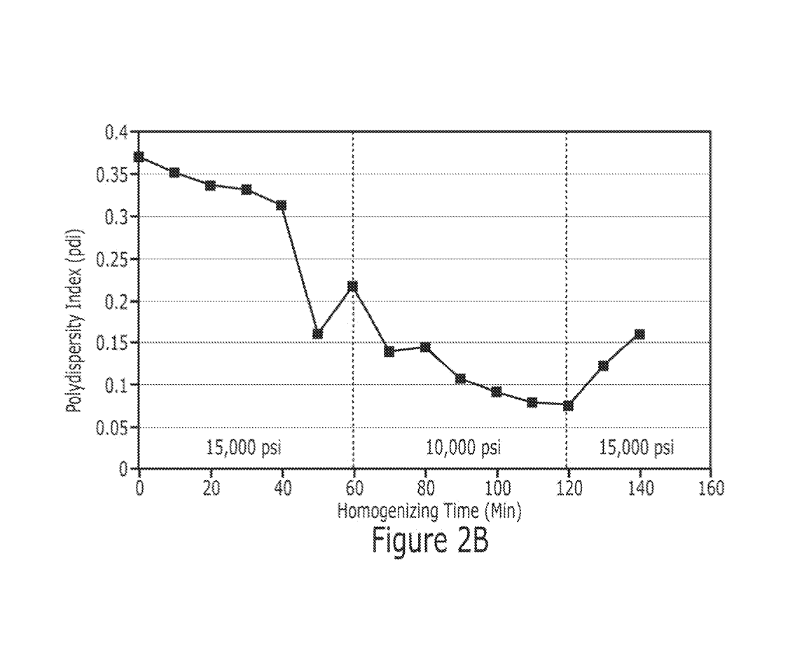

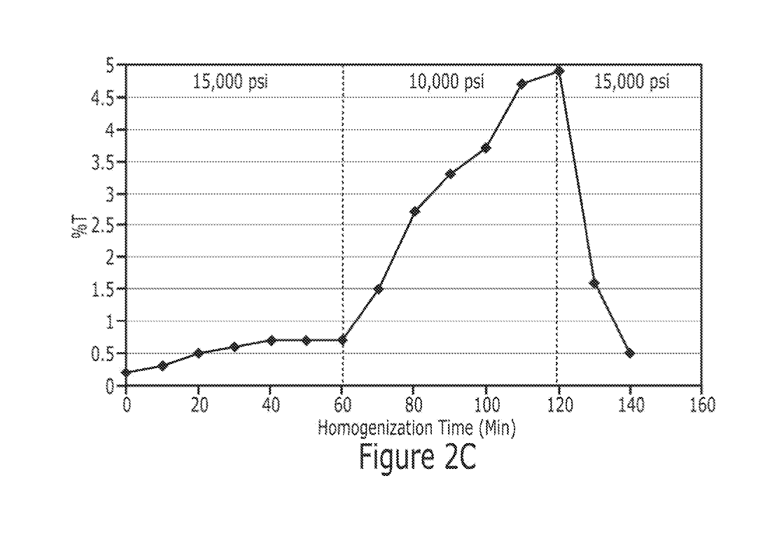

As shown in FIG. 2A, homogenization pressure of 15,000 psi (1034 bar) produced a rapid decrease in average diameter during the initial 20 minutes of processing and then a continued slower rate of decrease until 60 minutes. At that time, homogenization pressure was reduced to 10,000 psi (690 bar) and average diameter reached a stable minimum of about 82 nm. At 120 minutes processing time, homogenization pressure was increased to 15,000 psi, which resulted in a marked increase in average diameter.

As shown in FIG. 2B, polydispersity (PDI) decreased rapidly during, the initial 45 minutes of treatment at a homogenization pressure of 15,000 psi and then climbed during treatment until 60 minutes. At that time, homogenization pressure was reduced to 10,000 psi and PDI decreased steadily to reach a low at 120 minutes total processing time. At that time, homogenization pressure was increased to 15,000 psi and PDI rapidly increased during the time, the liposomes were subjected to this homogenization pressure.

As shown in FIG. 2C, %T rose slowly during 60 minutes of treatment at a homogenization pressure of 15,000 psi. When the homogenization pressure was then reduced to 10,000 psi, %T rapidly rose. At 120 minutes, when homogenization pressure was again raised to 15,000 psi, %T rapidly and markedly decreased.

These results establish that homogenization pressure is a result-effective variable in the homogenization and size reduction of liposomes. Utilizing a homogenization pressure of 10,000 psi provided rapid and sustainable size reduction of liposomes in a suspension. In contrast, a homogenization pressure of 15,000 psi was ineffective in size reduction and, in fact, homogenizing at this higher pressure effectively negated the homogenization that was obtained when processing at the lower 10,000 psi.

### Example 4

Size Reduction with Change in Homogenizing Pressure

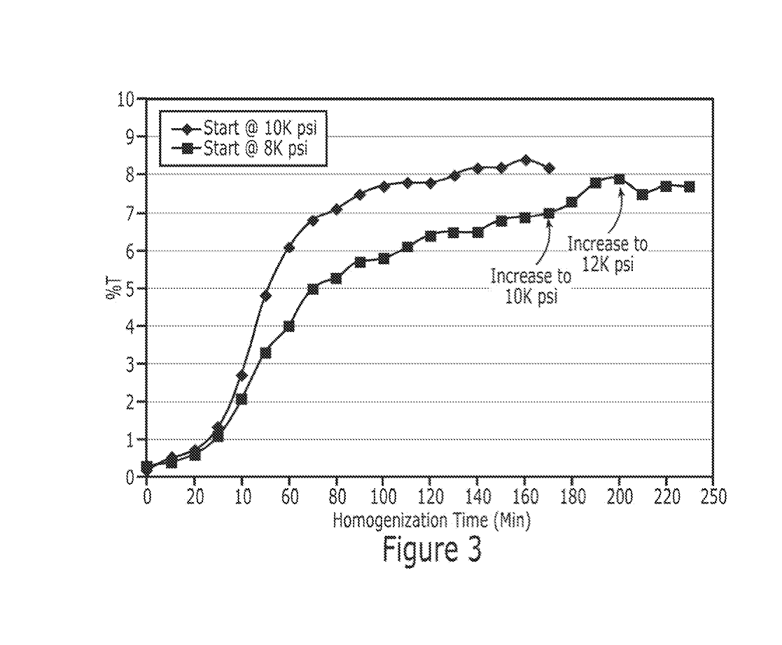

Pegylated liposome suspensions prepared according to Example 1 were subjected to CLP processing according to Example 2, except that homogenization was performed at a pressure of either 10,000 psi (690 bar) or 8,000 psi (552 bar). The results are shown in FIG. 3.

As shown in FIG. 3, both 10,000 psi and 8,000 psi curves show the same plot shape, the lower-pressure plot shows the change in %T occurring at a slower rate than occurred with the higher pressure. Additionally, when processing pressure was raised from 8,000 psi to 10,000 psi, the rate of change in %T increased, permitting the attainment of the final %T that was obtained in the suspension that was homogenized consistently at a pressure of 10,000 psi. Additional increase in homogenization pressure to 12,000 psi (827 bar) not only stopped further increase in %T but possibly caused a slight reduction in %T. These results indicate that, among homogenization pressures of 8000, 10,000 and 12,000 psi, size reduction of liposomes was optimal at a pressure of 10,000 psi.

### Example 5

Homogenization of Liposomes

Liposome suspensions made according to Example 1 were subjected to CLP processing according to Example 2 at a pressure of 10,000 psi for a duration of either 10 minutes or for a duration of 180 minutes. A duration of 10 minutes was utilized as this time is insufficient to obtain asymptotic values. A duration of 180 minutes was utilized as this time is sufficient to obtain asymptotic values.

The results are shown in FIGS. 4A and 4B. As shown in FIG. 4A, after 10 minutes of processing at a pressure of 10,000 psi, a polymodal size distribution of liposomes was obtained, with peaks at 93.02 nm, 901.4 nm, and 5,048 nm. Of these peaks, the largest peak, at 93.02 nm, had an intensity of 80.7%.

As shown in FIG. 4B, after 180 minutes of processing at this pressure, the distribution of peaks was uni-modal. Of special importance to note is that the largest peak had a narrower size distribution than did the largest peak obtained after only 10 minutes and that the largest peak also was at a lower size, 88.67 nm, than obtained after only 10 minutes.

### Example 6

Drug Loading of Liposomes

After homogenization, the liposomes were dialyzed with 10% sucrose solution via tangential flow filtration to remove external ammonium sulfate, leaving the liposomes suspended in the sucrose solution but maintaining ammonium sulfate internally within the liposomes. A solution of doxorubicin HCl (MicroBiopharm Japan Co., Ltd., Tokyo, Japan) in 10% sucrose was combined with the liposome suspension at 60° C. in order to encapsulate the drug within the liposomes.

While preferred embodiments of the invention have been described in detail, it will be apparent to those skilled in the art that the disclosed embodiments may be modified. It is intended that such modifications be encompassed in the invention. Therefore, the foregoing description is to be considered to be exemplary rather than limiting, and the scope of the invention is that defined by the following claims.

1. A method for size reduction of liposomes comprising subjecting a suspension containing the liposomes to a homogenization process and continuing the homogenization process for a time sufficient to obtain an asymptotic limit to percent transmittance through the suspension, wherein the method of size reduction of the liposomes in the suspension does not include extrusion, either prior to or after the homogenization process.

2. The method of claim 1 wherein the homogenization process utilizes a method selected from the group consisting of shear forces, pressure differential, and sonication.

3. The method of claim 2 wherein the method is pressure differential.

4. The method of claim 2 wherein the process utilizing pressure differential is piston-gap homogenization.

5. The method of claim 4 wherein the piston-gap homogenization is performed at a pressure of between about 5,000 psi and about 12,000 psi.

6. The method of claim 5 Wherein the pressure is between about 8,000 psi and about 11,000 psi.

7. The method of claim 6 wherein the pressure is about 10,000 psi.

8. The method of claim 4 which is by continuous-loop processing (CLP).

9. The method of claim 1 wherein the liposomes are coated with a substance that increases enhanced permeability and retention (EPR) of liposomes.

10. The method of claim 9 Wherein the substance is polyethylene glycol.

11. A method for making liposomes containing within a drug for administration into the body of a subject in need thereof comprising obtaining a suspension of liposomes that do not contain within the drug, subjecting the suspension containing the liposomes to a homogenization process and continuing the homogenization process for a time sufficient to obtain an asymptotic limit to percent transmittance through the suspension, wherein the method of size reduction of the liposomes in the suspension does not include extrusion, either prior to or after the homogenization process, and loading the drug into the liposomes.

12. The method of claim 11 wherein the drug is remote loaded into the liposomes.

13. The method of claim 11 wherein the drug is doxorubicin.

14. The method of claim 11 wherein the homogenization process utilizes a method selected from the group consisting of shear forces, pressure differential, and sonication.

15. The method of claim 14 wherein the method is pressure differential.

16. The method of claim 15 wherein the process utilizing pressure differential is piston-gap homogenization.

17. The method of claim 16 wherein the piston-gap homogenization is performed at a pressure of between about 5,000 psi and about 12,000 psi.

18. The method of claim 17 wherein the pressure is between about 8,000 psi and about 11,000 psi.

19. The method of claim 18 wherein the pressure is about 10,000 psi.

20. The method of claim 11 Wherein the homogenization is by continuous-loop processing (CLP).

21. The method of claim 11 wherein the liposomes are coated with a substance that increases enhanced permeability and retention (EPR) of liposomes.

22. The method of claim 21 wherein the substance is polyethylene glycol.
